(19) United States
(12) Patent Application Publication
(10) Pub. No.: US 2026/0076622 A1
(43) Pub. Date: Mar. 19, 2026

(54) A LIGHT WEIGHT AND/OR SMALL DEVICE FOR DETERMINING HEALTH CONDITIONS

(71) Applicants: VRIJE UNIVERSITEIT BRUSSEL, Brussel (BE); UNIWEB BVBA, Meise (BE)

(72) Inventors: Rudi TIELEMANS, Meise (BE); Ivan BAUTMANS, Vilvoorde (BE); Hugo Humerto Plácido DASILVA, Lisboa (PT)

(21) Appl. No.: 19/109,787

(22) PCT Filed: Sep. 7, 2023
(86) PCT No.: PCT/EP2023/074617

§ 371 Date: Mar. 7, 2025

(30) Foreign Application Priority Data
Sep. 9, 2022 (BE) 2022/5714

(51) Int. Cl. A61B 5/00; A61B 5/085; A61B 5/097; A61B 5/11

(52) U.S. Cl. CPC A61B 5/68; A61B 5/085; A61B 5/097; A61B 5/1107; A61B 5/7267; A61B 5/746; A61B 2562/0219; A61B 2562/0247

(57) ABSTRACT

The invention is in the field of measuring health conditions (for the purpose of assessing, monitoring and/or evaluating) in a human being based on one or more tests executed by said human and provides specific electro-mechanical devices, pneumatic components, systems (configurations) and/or arrangements of specific and specially configured electro-mechanical devices for such purpose, such health conditions or derivatives thereof being one or more selected from the group of vitality capacity, physical reserves, frailty, resilience, intrinsic capacity, fatigue, fatiguability, stress response.

### BACKGROUND OF THE INVENTION

Providing health conditions to a human being based on a test executed by said human are known in a laboratory environment.

In a typical setting the device used to perform a test (like a weightlifting device or a cycle ergometer) and the capturing of at least part of the test data is separated from such device and even not necessarily automated.

Typically said test devices are big, heavy, expensive and complex to operate.

Providing health conditions to a human being based on a test executed by said human in a laboratory environment are typically done by performing a plurality of tests, each test performed on a separate test device.

Providing health conditions to a human being based on a test executed by said human in a laboratory environment are typically done under supervision of trained people, who may give feedback during the tests, based on their observations, such that a better test result is achieved.

Providing health conditions to a human being based on a test executed by said human in a laboratory environment are typically done rather infrequently.

### AIM OF THE INVENTION

One of the aims of the invention is to be able to execute a test for determining health conditions by humans in other than said laboratory environments such as in the human her or his home context and/or doctor cabinets less equipped than a laboratory environment.

One of the aims of the invention is to be able to execute a plurality of tests for determining health conditions or derivatives thereof by use of a single test device. Note that each test may give already a primary health condition parameter but the combining the test results (optionally supplemented with additional data) may give improved and/or other health conditions, hence with such health conditions or derivatives thereof is meant being one or more selected from the group of grip strength, muscle fatiguability, neuromuscular strength, fatigue, respiratory muscle strength, oxygen saturation, electromyography, electroencephalography, balance, tremor, coordination, endurance, blood biomarker, bio-impedance, spectroscopy.

One of the aims of the invention is to be able to still provide adequate test results absent the presence of supervision.

One of the aims of the invention is to perform tests more frequently than in a lab environment and hence capture much more test data, which can be exploited for improving the determination and monitoring (status or changes) of health conditions, preferably in a very early stage.

### SUMMARY OF THE INVENTION

It is a first aspect of the invention to provide a test device, which is both used by a human to perform the test but also to capture the test data. In a preferred embodiment, said test device is a light weight, small (hand-held) device, optionally provided with some means for stand-alone operation (hence enabling guidance for the test).

It is a second aspect of the invention to provide an arrangement comprising the test device of the first aspect of the invention and a second device, capable to receive the test data from said first test device and capable of providing health conditions of said human based thereon. In a preferred embodiment, the second device is a typical (also small (hand-held)) device, customary used at home, like a smart phone or tablet. However note that alternative arrangements with more advanced user equipment like augmented reality glasses is possible.

In another alternative embodiment, the second device is a remotely hosted computer server, which the test device communicates with wirelessly or using cellular connectivity. In yet another alternative embodiment a plurality of the mentioned second devices are used, e.g. exploiting the advanced user equipment for guiding the test, the hand-held device for its easy of providing information while the server is used for more advanced computations and storage of other data (of other test persons) used in the training of models.

It is a third aspect of the invention to provide a test device which can be used for performing a plurality of tests.

In one embodiment of the invention of the third aspect is realized by providing within said test device plurality of sensors which are different in nature, like pressure, accelerometer, gyroscope, magnetometer, bioelectricity, bio-impedance, piezoelectricity, spectroscopy.

In another embodiment of the invention of the third aspect is realized by providing said test device as a fixed part and supplementary parts like blowing tube, hand grip bulb, electric sensors, spectroscopy dedicated to perform one of the tests of a sequence of tests.

Note that in the embodiments exploiting pneumatic based sensing an airtight circuit is used.

These two embodiments can be combined and can be combined with the first and second aspect and their preferred embodiments.

It is a fourth aspect of the invention to arrange said second device (smart phone or tablet and/or augmented reality glasses) to be capable of providing guidance for said human to execute the test based on already captured test data by said test device.

This aspect can be combined with any of the foregoing aspects and (preferred) embodiments.

It is a fifth aspect of the invention to provide the test data obtained via said second device into data analysis environments for analysis and improving the determining of health conditions, e.g. by improving the data processing of the test data and/or improving the one or more tests.

This aspect can be combined with any of the foregoing aspects and (preferred) embodiments.

The invention is further demonstrated for grip strength (GS) and muscle fatigability measuring with a system comprising of a rubber bulb that is wirelessly connected to a smartphone-based application, and a tele-monitoring platform.

In an exemplary embodiment system is comprised of a large rubber bulb, as used in a typical lab set-up as a MV, connected to an analog Honeywell TruStability HSCMANN100PGAA3 gauge type pressure sensor, with signal axial barbed port, 0-100 PSI measurement range, and accuracy of ±0.25% FSS BFSL (Full Scale Span Best Fit Straight Line). The sensor is connected to an analog-to-digital conversion (resolution=10-bit and sample frequency=100 Hz) and data transmission. The system was connected via Bluetooth to a smartphone with the related software application was installed.

### BRIEF DESCRIPTION OF THE DRAWINGS OF THE INVENTION

FIG. 1 shows a first test device and its relation to a second device

FIG. 2 shows an (hand-held) electronic device comprising: (i) a pressure sensor; (ii) means for externally communicating data signals of said pressure sensor; and (iii) a capable for mechanical connecting a plurality of measurement tools to the electronic device.

FIG. 3 shows the (hand-held) electronic device of FIG. 2 further comprising: (i) input means for electrically connecting one or more additional measurement tools to the electronic device.

FIG. 4 shows a typical time signal obtained with the device and shows the training of a network.

FIG. 5 shows pictures of an embodiment of the first device, the system or configuration of the device and one of the measurement instruments

FIGS. 6 to 8 shows more exemplary pictures of the device.

FIG. 9 shows an exemplary data signals captured by the device.

### DESCRIPTION OF THE INVENTION

### Description of the Invention Based on the Drawings

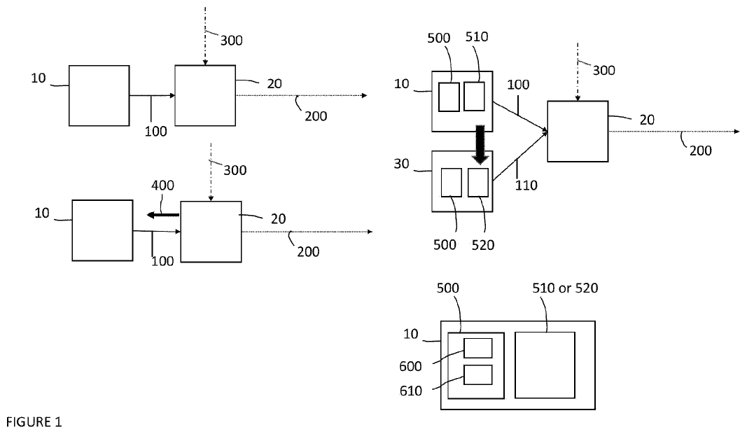

(FIG. 1 Top Left) The invention relates to the use of a first test device (10) by a human to capture test data (100) and to provide this to a second device (20), capable for inputting other data (300) and based on said test and/or said other data, providing health conditions (200) of said human. Said test device includes a microprocessor, telecommunication module and battery.

(FIG. 1 Bottom Left) The invention relates to the use of a first test device (10) by a human to capture test data (100) and to provide this to a second device (20), capable for inputting other data (300) and based on said test and/or said other data, providing health conditions (200) of said human, whereby said second device being capable of providing guidance (400) for said human to executing the test.

(FIG. 1 Top Right) The invention relates to the use of a first test device (10) by a human to capture test data (100) and to provide this to a second device (20), capable for inputting other data (300) and based on said test and/or said other data, providing health conditions (200) of said human, wherein said first test device is in essence reconfigurable in two configurations (10) and (30) in that it comprises at fix part (500) attached to either a first further part (510) (to form the first configuration (10)) or a second further part (520) (to form the second configuration (30)). The health conditions are derived from two subsequent tests, one obtained with the first configuration and one obtained with the second configuration. This embodiment can be combined with the embodiment of FIG. 1 Bottom Left in that in both said configurations guidance might be provided, whereby the guidance might be different per configuration.

The providing of data from said first to said second device is based on telecommunication means and preferably wireless. The inputting of other data can be by direct input to said second device but can alternatively also be supplied by telecommunication means and preferably wireless. The providing of said health conditions and/or guidance can be by direct output by said second device but can alternatively also be supplied by telecommunication means and preferably wireless to other auxiliary devices supporting this.

(FIG. 1 Bottom Right) The fix part (500) of the first test device (10) comprises one, preferably a plurality of sensors (600), (610), which are different in nature while the further parts (510) (520) are supplementary parts, dedicated to perform one of the tests of the sequence of tests. Given the plurality of sensors, the test data (100) comprises a plurality of sub datasets, which are each of a different nature, as they are captured by different sensors. 5

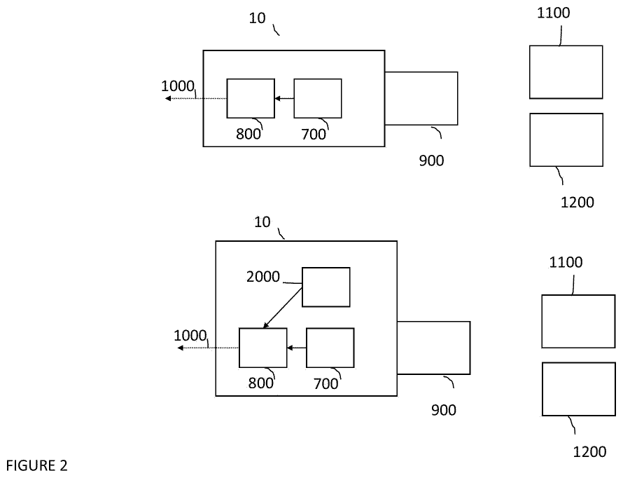

(FIG. 2 Top) shows an (hand-held) electronic device (10) comprising: (i) a pressure sensor (700); (ii) means for externally communicating (1000) data signals of said pressure sensor (800); and (iii) a connector (900) capable for mechanical connecting a plurality of measurement tools to the electronic device. In one embodiment (as measurement tool) an elastic pear (1100) is connected to said connector. In another embodiment (as measurement tool) a mouth piece (1200) is connected to said connector. The invention hence provides an arrangement of an (hand-held) electronic device (10) and at least said two measurement tools.

(FIG. 2 Bottom) shows the (hand-held) electronic device (10) of FIG. 2 Top further comprising: (i) an accelerometer (2000) connected to said means for externally communicating (1000) data signals. 15

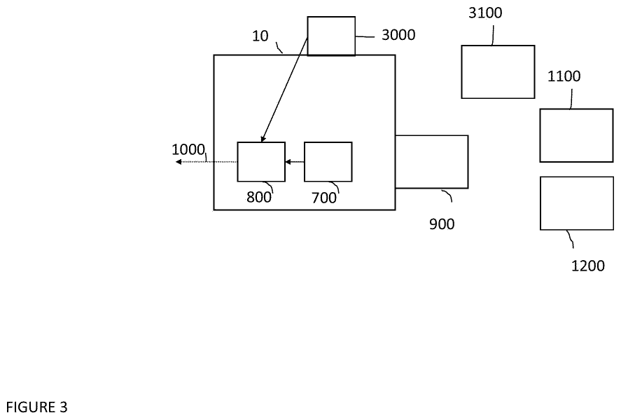

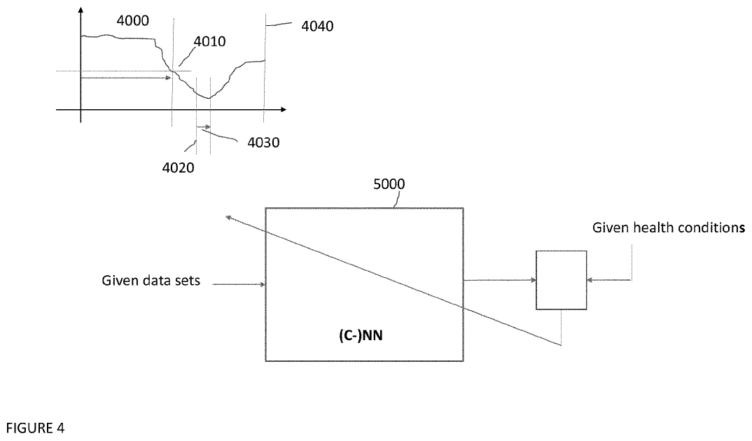

(FIG. 3) shows the (hand-held) electronic device (10) of FIG. 2 Top further comprising: (i) input means (300) for electrically connecting one or more additional measurement tools (3100) to the electronic device. In an embodiment (as measurement tool) one or more electrodes (connectable) is or are connected to said input means. In this embodiment the invention hence provides an arrangement of an (hand-held) electronic device (10) and at least said three measurement tools. (FIG. 4) (top) shows a typical time signal (4000). Obviously as this is running on digital equipment these are time discrete values. On the capture time signal, standard operations like averaging or smoothing can be applied to the extent that they do not disturb the features. In exemplary embodiment this can be the force put on the elastic pear by the human under test. (4010) shows a threshold which can be used to determine the time from the start of the test until the threshold is obtained. (4020) is the time of a feedback signal given which can be used to determine the time (4030) from the receipt of such feedback signal to actual effects observed in the time signal (e.g. no longer declining). (4040) is the end time of the experiment or test, which can also be dynamically determined, meaning based on the test signal.

(FIG. 4) (bottom) shows the training of a network.

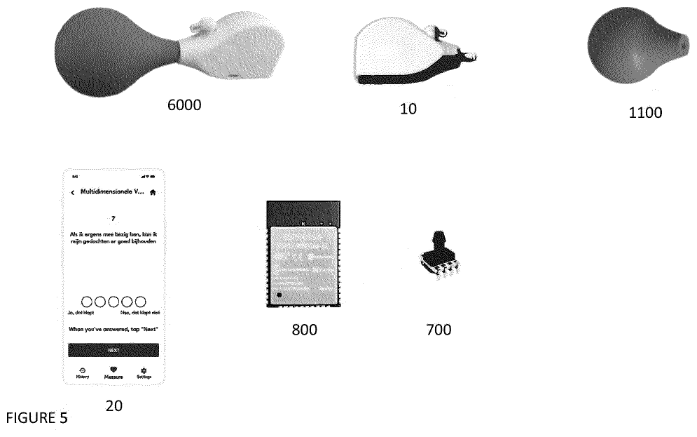

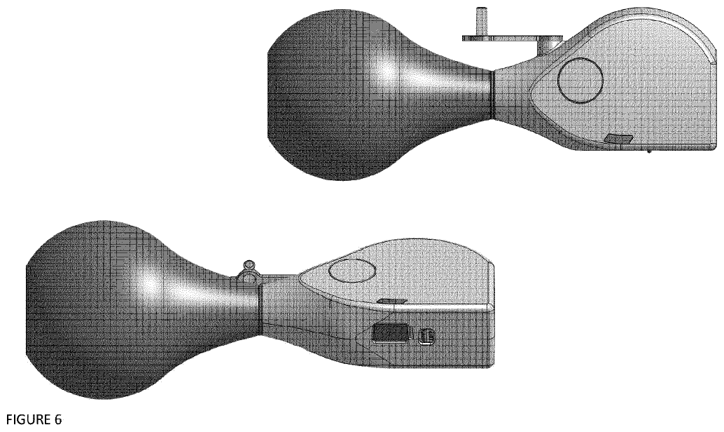

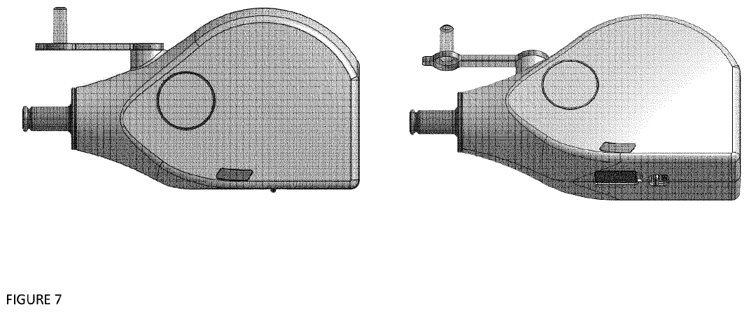

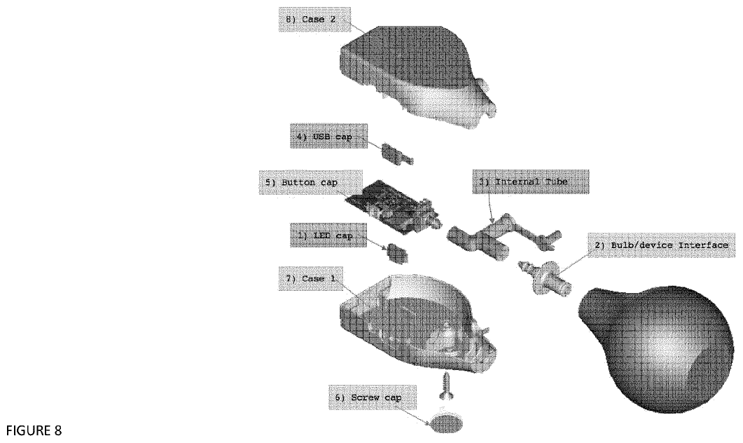

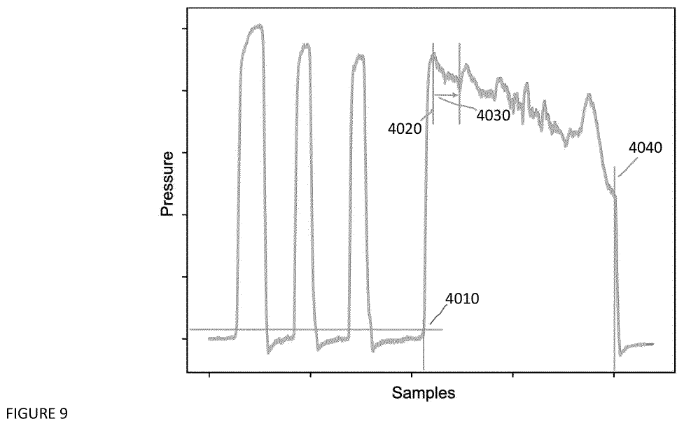

(FIG. 5) shows pictures of an embodiment of the first device (10), the system or configuration (6000) of the device (10) and one of the measurement instruments, in particular the elastic pear (1100), an example of a suitable pressure sensor (700), means for externally communicating data signals of said pressure sensor (800) and a typical display of the second device (20).

### Further Description of the Hardware Part

The invention provides a pneumatic component and an electro or electromechanical test device, capable to measure the force exhibited by a person, to measure the breathing condition of a person, to measure tremble or other movements exhibited by a person and to measure parameters related to the muscles of a person. The device is arranged in that at least two of said measurements set can be measured simultaneously and in a preferred embodiment at least three of said measurements set can be measured simultaneously.

In an embodiment of the invention provides a system or arrangements of material comprising the just described electro or electromechanical test device and (as measurement tool) an elastic pear (connectable) connected to said connector (for measuring the force exhibited by a person on said elastic pear via a pneumatic measurement by said pressure sensor) and/or (as measurement tool) a mouth piece connected to said connector (for measuring the breathing condition of a person exhaling in said mouth piece by use of said pressure) and/or (as measurement tool) one or more electrodes (connectable) connected to said input means (for measuring parameters related to the muscles of a person).

In an embodiment of the invention provides a system or arrangements of material comprising the just described electro or electromechanical test device and (as measurement tool) an elastic pear (connectable) connected to said connector (for measuring the force exhibited by a person on said elastic pear via a pneumatic measurement by said pressure sensor) whereby (as measurement tool) one or more electrodes are provided on the rubber bulb itself and (connectable) connected to said input means (for measuring parameters of a person).

Note that form factor of the electronic device may be selected to fit within the an elastic pear (connectable) connected to said connector.

The invention exploits the insight that capturing complementary data signals simultaneously gives a rich insight in the health condition and combines this with the technical consideration on possible re-use of part of the sensors (in particular the pressure sensors) and/or the technical requirements related to the (mechanical) connections to be made with the human interface (like the elastic pear or mouth piece). The invention carefully considers the to be executed test, test sequence and the reconfiguration (change of configuration) requirements.

The device is provided with (light weight, in the sense of not disturbing the experiments) energy storage means like batteries, at least sufficient to power the sensor and means for externally communicating data signals of said pressure sensor and/or any other electronic means within the device requiring energy.

Note that the input means (like USB connections) for electrically connecting one or more additional measurement tools, which is an embodiment of the invention just support a wired connection, may also be a means for internally communicating data signals towards said device, hence based on telecommunication, possibly wireless.

While the invented device is described in more detail in an arrangement comprising the device and a (hand-held) communication device, arranged for receiving data signals from said means for externally communicating data signals, this does not prevent having of (some) display capability of the device itself.

While the invented device is described in more detail in an arrangement comprising the device and a (hand-held) communication device, configured for determining health conditions of a person based on the received signals, this does not exclude that the invented device has (some) compute capabilities determining health conditions of a person based on the captured signals itself, even up to a full autonomous version.

### Further Description of Data Processing Part

The invention relates to deriving or determining or estimating one or health conditions parameters (jointly called the health condition) from one or more measurements.

In an aspect of the invention, with measurements is not meant a single value per measurement in time but a sequence of values, in that time signal is recorded. The deriving or determining or estimating one or health conditions parameters is then based on features of such time signals. To avoid that the recording and/or the digitalization thereof affects the time signal actually recorded to an extent that the deriving or determining or estimating one or health conditions parameters also get negatively affected, precautionary measures in terms of operational parameters on hardware or software level must be taken, for instance in ensuring that the sample frequency is high enough to avoid such negative effects. To be on the safe side, one may select the Nyquist frequency of the one or more time signals but one may also carry out dedicated experiments to choose a frequency in view of the features on needs to preserve.

In an aspect of the invention, based on the measurements and hence during the test, a feedback signal for the person under test is generated such that the person can adapt his efforts during the test. In this aspect of the invention deriving or determining or estimating one or health conditions parameters a feature of the measurements relates to the recorded response relative to such feedback signal.

In an aspect of the invention, it is recognized that the duration of the test is important for deriving or determining or estimating one or health conditions parameters in an accurate way. However lengthy tests are also not realistic. The invention generates an end signal to terminate the test based on the measurements.

In an aspect of the invention, it is recognized that the deriving or determining or estimating one or health conditions parameters mut be validated. The invention generates one or more validation signals based on the measurements.

In relation to precautionary measures in terms of operational parameters, in a particular embodiment those take into account the feedback signal and/or end signal and/or validation signal generation also.

In an aspect of the invention a feature of the signal is the maximum signal strength (e.g. if the signal relates to a force measurement, the maximum force that a person can generate is reflected therein). Another feature of the signal can be the time after a certain threshold is obtained (e.g. in relation to the force measurement, at what time has the force diminished e.g. to half of the maximum value or any other preselected value). As mentioned preferably the time sequence is looked at and one or more features are derived or computed based thereon. In an example one may determine the area under the measured curve as a feature. The threshold-based feature and/or space (integration under the curve) feature can be exploited for generating the feedback signal and/or said end signal. The examples above are relatively easy features to determine and the contribution of the invention is to relate those to the one or more health conditions, by use of formulas, for instance CPV ratio is defined as grip work/fatigue, hence the set-up is done in a two phases approach in that features are defined and thereafter related to the health conditions. In an alternative approach, artificial intelligence or machine learning methods are used, wherein algorithms (like (convolutional) neural networks) are trained or learned to predict one or more health conditions from the time signal. In essence this is a one-phased approach as the first layers of such algorithms will learn to extract features while the layers thereafter link those to the one or more health conditions. Of course a hybrid approach with predefined featured combined with to be learned features is also possible. In another hybrid approach, part of the relation is predefined with formulas.

In an exemplary embodiment the (timing of the) feedback signal is taken into account in said learning and/or the health conditions prediction or inference. The feedback and/or end signal may be generated also by training algorithms.

In an exemplary embodiment other available data of the person under test (like age, sex, weight) are also fed into the training and/or used for interference.

As mentioned before one or more measurements are used. In a first set-up the one or more measurements relate to force, tremble or other movements aspects derivable from the accelerometer, optional with further electrical measurements are used in the determining the one or more health conditions in one or another way, including the artificial intelligence way mentioned before. In a second set-up the one or more measurements relate to breath (strength), tremble or other movements aspects derivable from the accelerometer, optional with further electrical measurements, used in a similar fashion. In a preferred embodiment data or test signals from both set-ups are used.

Such methods can be described as methods, carried out by a computer, of training a model for computing for said one or more data sets, health conditions, said model, inputted with data sets as recording by said device or systems described above and outputting one or more health conditions, said training of said model being based on (i) loading a plurality of data set (from different persons) and their related health conditions and (ii) training, by adapting the model parameters, the model, based on said loaded information by comparing the output provided by the model with the loaded information (for instance capture in the lab by use of Martin Vigorimeter (MV), standard analog handgrip system). In an embodiment of the invention said model is a neural network, optionally a convolutional neural networks, said training providing for weights of said neural network.

### Demonstration

The system is evaluated for GS and muscle fatigability (defined as Fatigue Resistance (FR=time until GS decreased to 50% of maximum during sustained contraction) and grip work (GW-area under the strength-time curve)).

As the device allows for advanced data processing, the GW_{measured} can be calculated by integrating the actual GS over time during the test:

$$G {W}_{m e a s u r e d} = {\sum }_{( FR 100 - 50 )} G S * t$$

with GW_{measured}=measured grip work (kPa*s), GS=grip strength (kPa), t=time-interval (at 100 Hz=0.01s).

Alternatively, for instance to compare with data from lab set-up not having the above capability, as for the analog MV measure of GW, the device can also compute another value being the GW_{estimated} using the following equation:

$$G {W}_{estimated} = 0.75 * GS max * F R$$

with GW_{estimated} being estimated grip work (kPa*s), GSₘₐₓ being the highest maximal grip strength reached during GSₘₐₓ and FR-test (kPa), FR being fatigue resistance (time(s) during which GS dropped to 50% of its maximum).

1-17. (canceled)

18. An (hand-held) electronic device comprising: (i) one or more pressure sensors (for enabling pneumatic measurements); (ii) means for externally communicating data signals of said pressure sensor; and (iii) a connecting means capable for mechanical connecting a plurality of measurement tools to the electronic device, such plurality of measurement tools including (as measurement tool) an elastic pear (connectable) connected to said connector for measuring the force exhibited by a person on said elastic pear (via a pneumatic measurement by said pressure sensor) and/or (as measurement tool) a mouth piece (connectable) (directly or indirectly via intermediary tubes) connected to said connector for measuring the breathing condition of a person (exhaling in) by use of said mouth piece (by use of said pressure) optionally said connecting means comprising: a plurality of connectors, each connector being capable for mechanical connecting at least one of said plurality of measurement tools.

19. The electronic device of claim 18, further comprising an accelerometer and/or gyroscope and said means for externally communicating data signals being adapted for communicating data signals of said accelerometer and/or gyroscope.

20. The electronic device of claim 18, further input means for electrically connecting one or more additional measurement tools to the electronic device and said means for externally communicating data signals being adapted for communicating data signals of said additional measurement tools.

21. The electronic device of claim 18, being adapted for capturing data samples at a frequency exceeding 50 Hz, preferably 80 Hz, more preferable 100 Hz and/or at least a 8-bit, preferably 12-bit digital representation.

22. A system comprising: the electronic device of claim 18; and (as measurement tool) an elastic pear (connectable) connected to said connector for measuring the force exhibited by a person on said elastic pear (via a pneumatic measurement by said pressure sensor) and/or (as measurement tool) a mouth piece (connectable) (directly or indirectly via intermediary tubes) connected to said connector for measuring the breathing condition of a person (exhaling in) by use of said mouth piece (by use of said pressure).

23. The system of claim 22, wherein said intermediary tubes being provided with means to bring the internal pressure to a predetermined pressure suitable for the measurement to be executed such as atmospheric pressure for the breathing test.

24. A system comprising: the electronic device of claim 20; and (as measurement tool) one or more electrodes (connectable) connected to said input means (for measuring parameters related to the muscles of a person).

25. An arrangement comprising the system of claim 22, further comprising: a (hand-held) communication device, arranged for receiving data signals from said means for externally communicating data signals; and configured for determining health conditions of a person based on the received signals.

26. The arrangement of claim 25, wherein said communication device, being arranged for delivering a feedback (alarm) signal, based on said received signals, during a measurement session.

27. A method for deriving one or more health parameters of a person using the system of claim 22, comprising the steps of: (i) capturing data signals from said systems or arrangements; and (ii) deriving said one or more health parameters from time variations in said data signals and/or patterns in said data signals.

28. The method of claim 27, comprising the step of delivering a feedback (alarm) signal, based on said data signals, during a measurement session and/or the step of delivering validation signals, based on said data signals, in particular based on the data signals from said accelerometer.

29. A method for deriving one or more health parameters of a person comprising the steps of capturing first data signals from a first pressure sensor of a system comprising the electronic device of claim 18 and an elastic pear connected to said connecting means for measuring the force exhibited by a person on said elastic pear; capturing second data signals from a second pressure sensor of a system comprising the same electronic device and a mouth piece connected to said connecting means for measuring the breathing condition of a person (exhaling in) by use of said mouth piece) and (ii) deriving said one or more health parameters jointly from said first and second data signals, optionally said capturing of said first data signals and second data signals are performed one after another and preferably then the first and second pressure sensor are the same, alternatively the capturing of said first data signals and second data signals are performed substantially simultaneously (wherein the first and second pressure sensor are the different).

30. The method of claim 29, wherein simultaneous for capturing said first and/or said second data signals, (separate from said first and second data signals or alternatively substantially simultaneously with one or both of those) capturing third data signals from said accelerometer of the electronic device and said deriving said one or more health parameters exploits these third data signals.

31. The method of claim 29, wherein separate from said first, second data, third signals or alternatively simultaneously with one or more of those, capturing fourth data signals from said electrodes connected to said electronic device and said deriving said one or more health parameters exploits these fourth data signals.

32. The method of claim 29, wherein said deriving said one or more health parameters being based on a trained model.

33. A method, carried out by a computer, of training a model for computing for said one or more data sets, health conditions, said model, inputted with data sets as recording by said device or systems described above and outputting one or more health conditions, said training of said model being based on (i) loading a plurality of data set (from different persons) and their related health conditions and (ii) training, by adapting the model parameters, the model, based on said loaded information by comparing the output provided by the model with the loaded information.
